(19) United States
(12) Patent Application Publication
(10) Pub. No.: US 2008/0281206 A1
(43) Pub. Date: Nov. 13, 2008

(54) Ultrasound Measurement System and Method

(76) Inventors: Stewart Gavin Bartlett, Rose Park (AU); Roger Michael Costello, Winthrop (AU)

Correspondence Address: Intellectual Property Dept.;Dewitt Ross & Stevens SC, 2 East Mifflin Street, Suite 600, Madison, WI, 53703-2865, US

(21) Appl. No.: 12/092,599

(22) PCT Filed: Nov. 7, 2006
(86) PCT No.: PCT/AU06/01658

§ 371 Date: Jul. 17, 2008

(30) Foreign Application Priority Data
Nov. 7, 2005 (AU) 2005906152

(51) Int. Cl. A61B 8/00

(52) U.S. Cl. 600/459

(57) ABSTRACT

An ultrasound measurement system including a handheld display and processing means, an ultrasound transducer, a processing means of a substantially similar weight to the handheld display and processing means, and a transmission cable interconnecting the handheld display and processing means with the ultrasound transducer and processing means, the cable being of sufficient length to provide a means to mechanically locate the system around the neck of a user.

### FIELD OF THE INVENTION

The present invention relates to a low cost and efficient medical ultrasound imaging, measurement and recording system with a configurable interface that supports a variety of medical ultrasound probes.

### BACKGROUND OF THE INVENTION

Ultrasound was first investigated as a medical diagnostic imaging tool in the 1940's. George Ludwig was the first scientist to use amplitude mode (A-mode) ultrasound to detect foreign bodies in tissue. This is described in the report by Ludwig et al., “Considerations underlying the use of Ultrasound to detect Gallstones and Foreign Bodies in Tissue”, Naval Medical Research Institute Reports, Project #004 001, Report No. 4, June 1949. In the early 1950's Wild and Reid constructed a B-mode scanning system using a mechanically mounted rotating transducer, described in Wild, J. J. and Reid, J. M. (1952) “Application of echo-ranging techniques to the determination of structure of biological tissues”. Science 115:226-230 (1952). Ultrasound technology developed significantly in the 1960's with the development of articulated arm B-mode scanners by Wright and Meyerdirk (U.S. Pat. No. 1970000062143). Articulated arm scanners, also known as static mode scanners, connect the ultrasonic transducer to a moveable arm, with movement of the arm mechanically measured using potentiometers. Static mode ultrasound scanners were in wide use until the early 1980s. The static mode scanners were large cumbersome devices, and the techniques used are not readily suited to a handheld ultrasound system.

In the mid 1970's real-time scanners were developed where an ultrasonic transducer was rotated using a motor. Krause (U.S. Pat. No. 3,470,868—Ultrasound diagnostic apparatus) describes an invention where a motor rotates an ultrasonic transducer in order to produce images in real-time. The clinical usefulness of such real-time B-mode scanners is outlined in the article by J. M Griffith and W. L Henry titled “A sector scanner for real-time two-dimensional echocardiography”. Circulation 49:1147, 1974. The nature of these devices, as well as the motor driving circuitry, adds size, power consumption, and cost to the device. Additionally, the motor itself and associated moving parts reduces the reliability of the device.

The further development of ultrasound resulted from developments in electronic beam steering transducers. Wilcox (U.S. Pat. No. 3,881,466) describes an invention consisting of a number of electronic crystals where the transmitting pulse can be delayed in sequence to each crystal and effect an electronic means to steer the ultrasound beam. The basic technique is still in wide use today, with nearly all modern medical ultrasound equipment using an array of ultrasonic crystals in the transducer. The early designs used at least 64 crystals, with modern designs sometimes using up to a thousand crystals or more.

Electronic beam steering removes the need for a motor to produce real time images, but the cost of producing transducers with arrays of crystals is high. The transducers are usually manually manufactured, with the channels having excellent channel to channel matching and low cross-talk. The probe cost is not an important factor in state-of-the-art ultrasound diagnostic systems, as the overall equipment cost is several times the probe cost. The power consumption for electronic systems is also high, and is generally proportional to the number of channels being simultaneously operational.

Much of the prior art in ultrasound technology is directed to improving the performance of ultrasound systems enabling them to be used for an ever increasing range of diagnostic applications. The result has seen significant advances in ultrasound systems with transducers using ever increasing numbers of crystals, and host systems with ever increasing processing power. The result has seen systems with 3D and real-time 3D (or 4D) capability.

Some manufacturers have focussed on producing systems which are more portable than the large and bulky systems used in radiology clinics and large hospitals. Sonosite have developed products able to be hand-carried (U.S. Pat. Nos. D461895, 6,575,908) using transducer with arrays of crystals. The cost and power consumption of the Sonosite systems is far less than the large cart based systems, but still too expensive for most primary care physicians. Chiang et al (U.S. Pat. Nos. 5,590,658, 5,690,114, 5,839,442, 5,957,846, 6,106,472) disclose a system with a beamforming array using charge domain processing connected to a host processing unit via a high speed interface. The preferred embodiment connects to a laptop computer, however those skilled in the art would understand the device could be connected to a handheld processing system. Halmann et al (U.S. Pat. No. 7,115,093) of General Electric disclose a similar device, specifically intended for use with a handheld processing system, which uses digital beamforming. However, both products still consist of expensive and power hungry multi-element transducer arrays resulting in a costly imaging system. Other hand-carried ultrasound systems are available from General Electric (Logiqbook family) and several other vendors, with a common characteristic of the devices being their inclusion of a multi-element transducer and a laptop sized processing system.

The hand carried ultrasound systems are improving in performance and are able to be used in diagnostic procedures only a short time ago limited to the larger cart based ultrasound systems. Sonosite claim the Micromaxx hand carried unit “represents the technology crossover point between hand-carried ultrasound and larger, high-performance, cart-based systems.” The trend has been for hand-carried ultrasound to improve where it can perform most of the diagnostic functions currently performed by more expensive cart based systems. The result is an increase in the cost of hand-carried systems, rather than a decrease.

Several inventors have investigated methods of reducing the cost of the transducers, although not necessarily for use with a handheld ultrasound system. Sliwa and Baba (U.S. Pat. No. 5,690,113) proposed a system where a stationary ultrasound transmitter coupled with position and orientation sensing circuitry are combined to form an inexpensive ultrasound probe. The system claims a non-real time ultrasound system consisting of either untethered probes with wireless communications or a tethered probe with an electromagnetic receiver mechanically coupled to the probe, and a separate electromagnetic transmitter providing a reference position signal. The probe could be manufactured cheaply enough to be disposable, reducing requirements for a sterilisation procedure between examinations and is especially suited to intra-uterine examinations. The requirement for the tethered transducer to have a separate stationary electromagnetic transmitter is well suited to cart or desk based systems, where the host processing unit does not move, but is not suitable for handheld systems where the host processing unit is moving. The requirement for a wireless communications system in the probe increases cost and power consumption, requiring additional components for the wireless communications system and a separate battery for the ultrasound probe.

Hunt et al broadly disclose an invention (U.S. Pat. No. 6,780,154) consisting of a segmented ultrasound system consisting of an ultrasound processor and transducer connected to a wireless handheld computing device. The ultrasound processor and transducer construct an image and wirelessly communicate the image to a display device in non-real time. The limitation of the invention is no low cost method is proposed to construct the ultrasound image, with the preferred embodiment being a 64 channel array. The system also requires a separate battery supply for the ultrasound processor and transducer, and incurs the overhead of the wireless communications scheme in power consumption limiting the battery life and utility of the device.

There is a need to improve on the prior art by constructing a handheld ultrasound system of low power consumption, low cost, low weight, of small size, and easy to use such that it can be used by primary care physicians.

### SUMMARY OF THE INVENTION

In accordance with a first aspect of the present invention, there is provided an ultrasound measurement system including: a handheld display and processing means; an ultrasound transducer and processing means of a substantially similar weight to the handheld display and processing means; and a transmission cable interconnecting the handheld display and processing means with the ultrasound transducer and processing means and being of sufficient length to provide a means to mechanically locate the system around the neck of a user.

Preferably, the handheld display and processing means includes a primary user input means and the ultrasound transducer and processing means includes a secondary user input means. Preferably, the primary user input means consists at least of a scroll wheel and push activated buttons, and the secondary user input means consists of a scroll wheel and push activated buttons. Preferably, the system also includes an ultrasonic transmit and receive means, and a position and orientation measuring means in order that the received ultrasound signals can be displayed in spatial register with each other. Preferably, the ultrasound transducer means further includes a non-volatile memory for storing position and orientation calibration data.

Preferably, the ultrasound transducer means includes a means for processing the position and orientation data and the calibration data and producing normalized position and orientation data. Further, the display and processing means can comprise a microphone and software means for recording user voice (dictation). The display and processing means can incorporate a communications means for connecting and sending recorded data to/from other systems for importing or exporting patient data. The display and processing means can include an integrated camera for recording images. The ultrasound transducer and processing means can include a gel dispensing means with a replaceable gel cartridge.

Preferred embodiments broadly disclose novel systems in which ultrasonic measurement and imaging can be conveniently performed with less complexity and cost than previously available devices. The preferred embodiment devices possess a range of novel characteristics whereby the cost of medical ultrasound scanning is significantly and advantageously reduced and which also enhances the ease of use and convenience of their operation to the level at which they are operable by a primary care physician.

Preferred embodiments of the invention include a handheld display and user input host system connected to an ultrasound transducer via a cable. The handheld display system and the ultrasound transducer system are manufactured to be of similar volume and mass, facilitating a balanced load when the system is carried around a user's neck or over a user shoulder. The systems and cable are also of a size to be conveniently folded and placed in a user's pocket.

The ultrasound transducer system consists of one or more elements for transmitting and receiving ultrasonic waves with associated transmission circuitry and receiver amplifiers. The receiver circuitry includes analog to digital converters for converting the electrical representations of the received ultrasonic energy to digital data. The ultrasound transducer system also contains a controller for communicating with the host system, controlling operation of the ultrasound apparatus, and accepting user inputs from local mechanical or electrical switches and user input means. A preferred embodiment also contains circuitry for measuring the orientation and/or position of the transducer relative to a starting point or external reference, a temperature sensor, and a means to store local calibration data. The position/orientation measurement data is processed with the calibration data according to the temperature and input, and combined with the ultrasound data before being transmitted over the cable to the host system, enabling a position measurement system of high accuracy without the host system being aware of the means of position measurement. The position and orientation measurement allows an ultrasound transducer where the transmission pulse is transmitted in a fixed relative position to the ultrasound transducer, but moved in space by the user moving the probe.

The ultrasound transducer system can include an ultrasound gel storage and dispensing system, removing the requirement to carry a bottle of ultrasound gel, and a camera, for recording scan locations.

The host processing and display system is of a size able to be conveniently held and controlled using a single hand. In a preferred embodiment, the processing and display system can be held in one hand and all functionality controlled using the users thumb. The second hand is free to hold and manipulate the ultrasound transducer. Alternatively, the host processing and display system can be mounted on a users arm using a strap freeing the first hand for other use. The second hand is free to hold and manipulate the ultrasound probe, and use the ultrasound probes secondary user input means to control the basic ultrasound functionality. The system can be configured to use position and orientation measurement circuitry in the ultrasound unit to generate user interface position information for “mouse” type operation.

The host processing and display system could advantageously contain communications components such as those enabling wireless network communications, and software enabling the interfacing to host computers or servers containing medical records databases, providing a simple and convenient means for transferring patient data to an electronic records system.

### BRIEF DESCRIPTION OF THE DRAWINGS

A preferred embodiment of the invention will now be described, by way of example only, with reference to the accompanying drawings in which:

FIG. 1 is a diagrammatic representation of the device.

FIG. 2 illustrates a user using the device.

FIG. 3 illustrates a user with the device resting around their neck.

FIG. 4 is a schematic diagram of one form of the preferred embodiment of the ultrasound system.

FIG. 5 is a schematic diagram of one form of the field programmable gate array (FPGA) utilised in the ultrasound system.

FIG. 6 is a schematic diagram of a second embodiment form of the ultrasound system.

FIG. 7 is a sectional view illustrating details of the ultrasound gel dispenser.

FIG. 8 is a schematic diagram of one form of implementation of the host display and processing unit.

### DESCRIPTION OF PREFERRED AND OTHER EMBODIMENTS

The background art provides several devices possessing unwieldy modes of operation. There is a need to integrate more fully the processing, recording, communication, display and control of ultrasound equipment and to reduce its cost and operational complexity such that it can be used by primary care physicians.

The preferred embodiment broadly disclose novel systems in which ultrasonic measurement and imaging can be conveniently performed with less complexity and cost than previously available devices. The preferred embodiment devices possess a range of novel characteristics whereby the cost of medical and veterinary ultrasound scanning is significantly and advantageously reduced and which also enhances the ease of use and convenience of their operation to the level at which they are operable by a primary care physician.

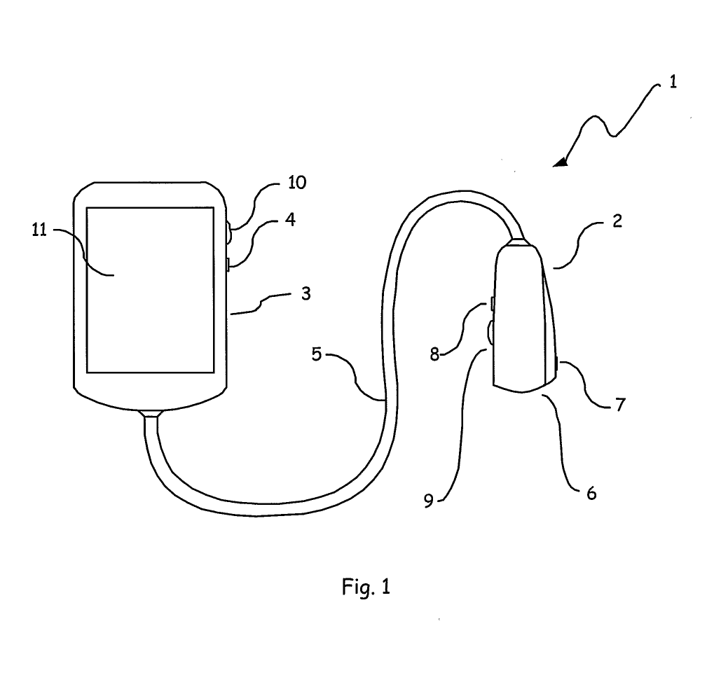

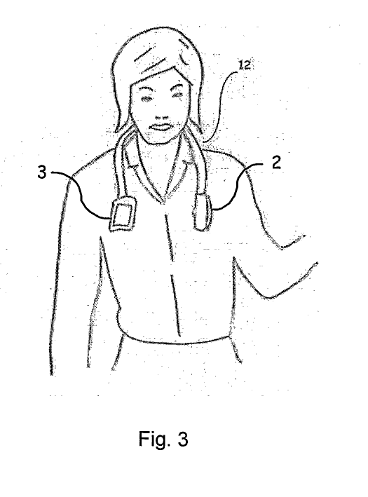

According to the invention there is provided an ultrasonic measurement and imaging system. An example embodiment is illustrated in FIG. 1. The system illustrated by 1 comprises a handheld display and processing system (3) connected to an ultrasound system (2) via a cable (5). The handheld display and processing system (3) and ultrasound system (2) are designed to be of substantially equivalent mass, enabling the system to be conveniently stored around a users neck, enhancing the portability of the device. An example of a user 12 implementing this mode of carriage is illustrated in FIG. 3. The ultrasound system contains an ultrasound tranducer or transducers (6) and a means for storing and dispensing ultrasound gel (7) removing the requirement for a user to carry an addition ultrasound gel dispenser.

The system is typically used by a user for an examination of a patient. The first phase is for setting up patient details. The second phase is ultrasound operation, with the user performing rudimentary user input such as selecting settings, and starting and stopping ultrasound scanning. The final phase is analysis and storage of the collected ultrasound data. To facilitate the different phases of examination, a variety of user input means are provided.

The handheld display and processing system (3) provides a scroll wheel (10) and a button user input means (4) to allow control of most operations.

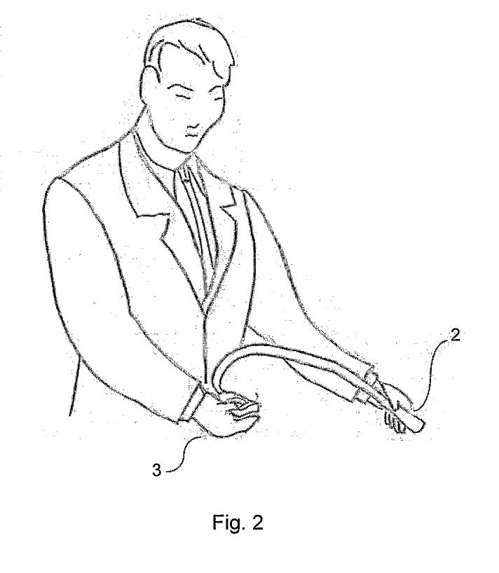

As illustrated in FIG. 2, the user input means (4) can be operated by a user's thumb or finger when the device (3) is comfortably resting in the user's hand, freeing the second hand to hold and control the ultrasound part of the system (2).

Alternatively, the handheld display and processing system can be mounted on a user's arm using a separate detachable strap/mounting means, freeing the corresponding hand for use in medical procedures such as ultrasound guided vascular procedures. For this operation, the ultrasound system (2) includes a secondary user input means (8 and 9) to control the handheld display and processing system (3).

The above described user input means are suitable for use during operation of the system such as during a patient examination. The first phase and third phase usually are before or after a patient has been examined, and therefore alternative more efficient text input means are provided.

The embodiment provides a stylus with a touch screen 11, and a Bluetooth interface enabling the use of wireless keyboards or input devices. A microphone in conjunction with a “Dictaphone” application can be used for voice recording. An alternative embodiment omits the touch screen but provides a means for interpreting position and orientation measurements in the ultrasound system (2) as part of user input, enabling the ultrasound system (2) to provide a positional (or mouse) style of user input.

Turning now to FIG. 2, there is illustrated a schematic block diagram of the components of the ultrasound system 2. The preferred embodiment of the ultrasound system (2) comprises an ultrasound transducer (13) for transmitting and receiving ultrasound energy in a fixed position relative to the housing. The ultrasound housing can be moved freely by the user with a means provided to measure the relative position and orientation of the ultrasound housing to a starting position. By capturing the received ultrasound energy, and the relative position of the ultrasound housing, the system can recreate a B-mode ultrasound image. The preferred embodiment uses a position and orientation measurement sensor (19) requiring few or no moving parts, such that the embodiment is less affected by reliability issues inherent in prior art which use a motor to move the transducer. A simple system uses solid state gyroscopic circuitry or an arrangement of accelerometers for measuring angular velocity in order to determine the orientation of the ultrasound system relative to a starting point. The inclusion of multiple accelerometers enables displacement to be measured.

A system with three accelerometers and three solid state gyroscopes can measure position and orientation for full 3 dimensional resolution. Much of the prior art discusses the drift problems inherent in accelerometer systems, however this problem is negated by the typical use of the present system. Typically, in use, a user places the ultrasound probe at the location where a scan is required, pointing at the object to be imaged. The user presses a button as part of the user input mechanism (22) to indicate a scan is to begin and holds the probe still. The system provides either audible or visual feedback to indicate a calibration has been successfully completed, and the user sweeps or moves the transducer through the required position and orientations. A scan occurs quickly and thereby limits the drift of the position and orientation system to a level within the systems resolution.

The transducer means (13) consists of one or more sensors for the transmission and reception of ultrasonic signals. For low cost, a single ultrasonic transducer element is provided, with focussing implemented by an acoustic lens or mirror system. Improvements to the system can be achieved at the expense of cost by adding additional transducer elements for transmit and receive operations.

The embodiment provides an ultrasonic system comprising a transmitting section (14) which generates one or more signals which stimulate the transducer means (13) to transmit ultrasound into the body of the patient, a diplexer (15) to protect the receive circuitry during transmission, and a receiving section which converts the ultrasound energy into electricity via the transducer (13) and amplifies (16) the electrical representation of the ultrasonic signals returned from the patient's body via a combination of reflection and refraction. The amplifier (16) typically can include a time-gain compensation amplifier where the gain is increased according to elapsed time from a pulse transmission. The amplified electrical signals are converted to a digital format by an analog to digital converter (17). The transmission of the ultrasound pulse can be initiated by a timing system implemented in a FPGA (18), which can also initiate a measurement of the housing position and orientation via position sensor (19) and temperature via temperature sensor (24). The timing system can be configured to only generate transmission of ultrasound pulses after the position and orientation sensor (19) has detected a change in position greater than a predefined threshold, thus minimising the amount of ultrasound energy and battery power used in the collection of an ultrasound scan. The position and orientation measurement means (19) also has its signal converted to a digital format by analog to digital converters (20) if required.

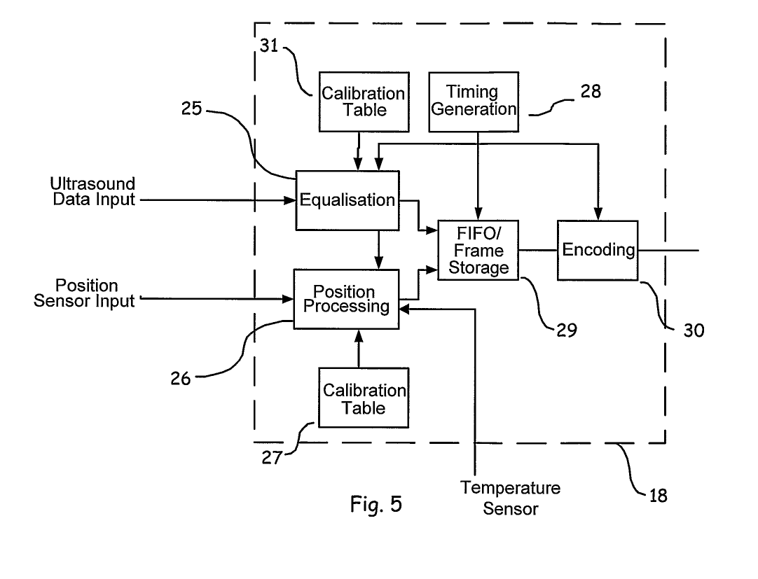

The FPGA (18) processes the position and orientation data to convert the information to a reference format, combines the data with the captured ultrasound data associated with the same measurement, and transmits the combined information via the interface (23) to the handheld display and processing system for further processing and display. A functional block diagram of the FPGA unit is displayed in FIG. 5.

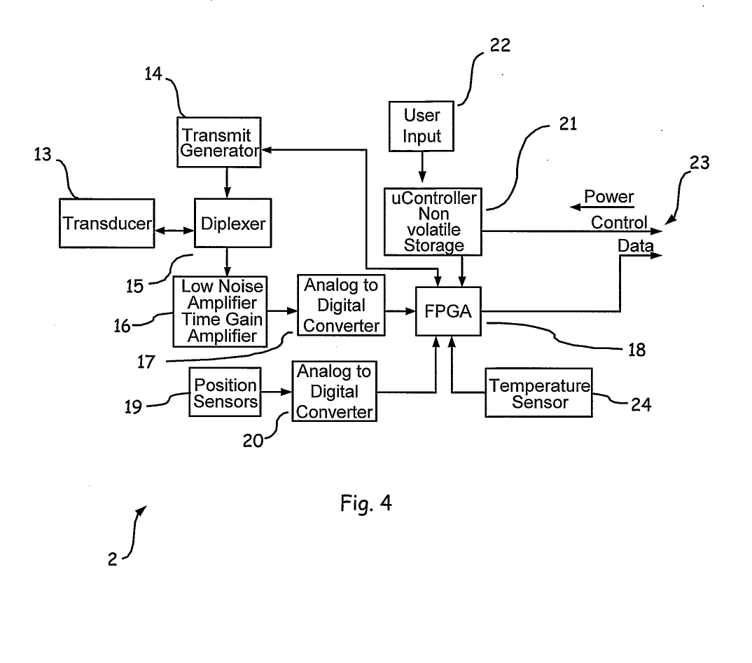

A systems microcontroller (21 of FIG. 4) can store calibration data for the position and orientation system and the Ultrasound Transducer, which are loaded into corresponding tables 27, 31 in the FPGA 18, enabling an increase in accuracy of the system overall. The calibration data is transferred to the FPGA (18) whenever the ultrasound system is readied for ultrasound scanning, and included in the processing of each individual position measurement. The calibration storage tables 27, 31 provide for the storage of calibration data on the probe enabling a consistent interface format regardless of probe design and construction (i.e. regardless of the arrangement and type of position and orientation means). In one embodiment, the calibration storage table is used in conjunction with a field-calibration process wherein a standard phantom is temporarily attached to the ultrasonic probe while the user instigates a calibration process, the results of which are stored in the calibration storage table 27.

Returning to FIG. 4, it is noted that the ultrasound system includes the secondary user input means (22) for controlling system operation. This user input means is preferably a scroll wheel with integrated button, and a separate button, implemented using either mechanical switches or any other technique well known and disclosed in the prior art. The ultrasound system decodes the user input 22 which is fed to the microcontroller (21). Any sort of modern microcontroller can be used, with the MSP430 series from Texas Instruments providing low standby power consumption, a variety of communications protocols, and non-volatile storage. The microcontroller communicates with the handheld display and processing system via interface 23 using a simple communications protocol, with I²C being particularly well suited due to its multi-master capability.

Turning again to FIG. 5, there is illustrated the FPGA in more detail. The FPGA contains a timing generator (28) responsible for synchronising all aspects of the ultrasound transmission, reception, and processing. Memory for temporarily storing calibration data associated with the ultrasound transducer (31) and position and orientation measurement means (27) is provided in the FPGA. The ultrasound calibration data (31) can be used to normalise or equalise the received ultrasound data with respect to the transducer response by implementing a filter (25) before transmission to the handheld display and processing system. The position and orientation calibration data table (27) is used to normalise the measured position and orientation data and reduce nonlinearities in sensor performance resulting in a more accurate position and orientation measurement, using a pre-measured calibration data and appropriate environmental measurements such as temperature. The position and orientation data is combined with the ultrasound data in a first in first out (FIFO) memory (29), before encoding the data (30) into a communications protocol for serial transmission to the handheld display and processing system.

The incorporation of calibration means and processing of the calibration on the ultrasound system allows a standard interface to a host processor system wherein different transducer means can be physically exchanged without the need to alter or adjust the operation of the body of the equipment.

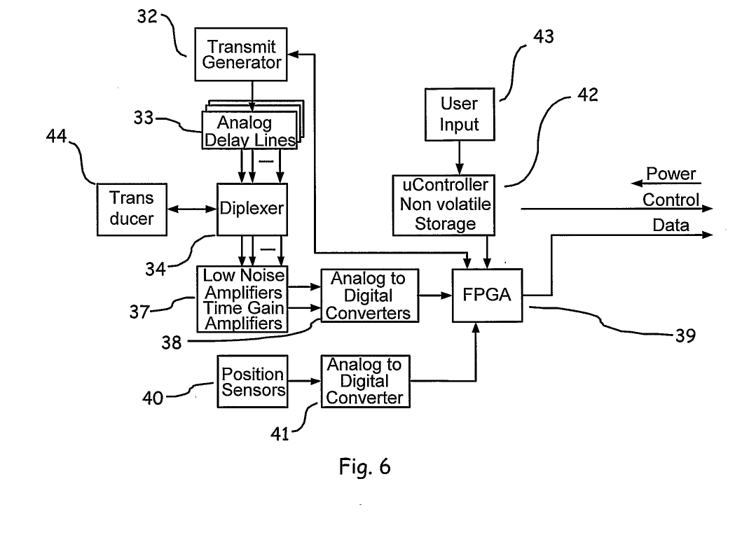

Various alternative embodiments of the Ultrasound system 2 are possible. FIG. 6 illustrates a functional block diagram of one alternative embodiment. The alternative embodiment of the ultrasound system 2 contains an annular transducer 44 with multiple transmit and receive elements. The pulse generated by the transmit generator (32) can be delayed by a set of analog delay lines (33) to vary the transmit focal length of the ultrasound pulse. A diplexer (34) protects the receive circuits from high transmit voltages. The received signals from the transducer can be amplified (37), converted to digital data (38), and combined with the position and orientation measurements (40 and 41) by the FPGA (39) before transmission to the handheld display and processing unit. User input means (43) and a microcontroller 42 having non-volatile storage can also be provided.

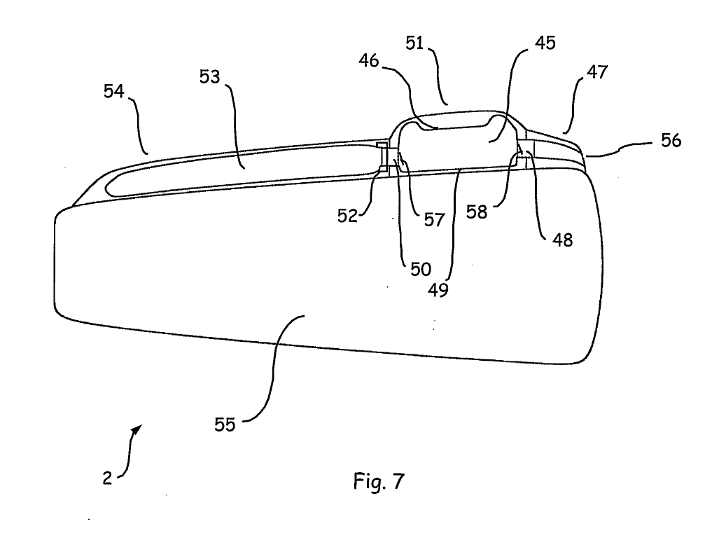

Turning now to FIG. 7, there is illustrated a schematic part sectional view through the transducer system 2. The electronic and transducer portion are stored within the lower cavity 55. Attached to the lower cavity is an ultrasound gel storage and dispensing means. The ultrasound gel dispenser includes a cartridge of gel (53) connected to a disposable pump (49). The gel cartridge is protected by a cover (54) which can be removed or detachable. The gel can be stored in a flexible packaging reducing cost, with a solid plastic connection means (52). The pump consists of a storage well (45) with a flexible membrane mechanism (46). The storage well has an input channel (50) providing a path for the gel to move from the storage packaging (53) to the storage well (45) via an input valve (57). The storage well (45) is also connected to an output channel (48) via an output valve (58). A flexible button cover (51) is pressed by the user which in term depresses the flexible pump membrane (46), forcing gel stored in the storage well (45) out of the output channel (48) via the output valve (58) and eventually out the output nozzle (56). When the button is released, the membranes (46) elasticity returns it to its previous shape, sucking gel from the storage packaging (53) into the storage well (45) via the input valve (57) and input channel (50).

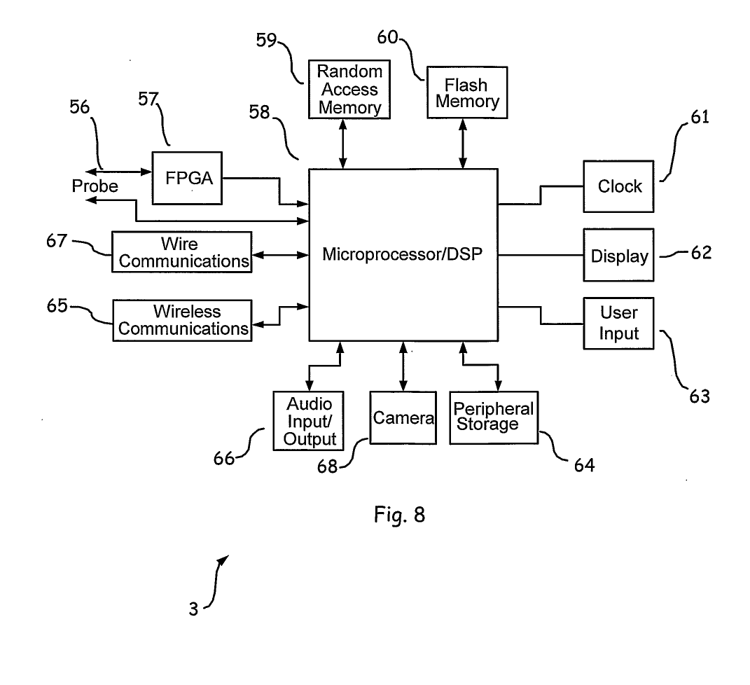

Turning now to FIG. 8 where there is illustrated a functional block diagram of the handheld display and processing system (3). The handheld display and processing system connects to the probe via a cable containing power, control communications, and data communications (56). The data input is connected to a FPGA (57), where the serial data is synchronised and decoded for reading by a microprocessor 58. The microprocessor is connected to volatile RAM storage (59) and non-volatile flash memory storage (60). The flash storage (60) contains program and operating system code, which is copied to and run from the volatile RAM storage (59). The display and processing system contains all or a subset of wired communications (67), audio input and output means (66), wireless communications means (65), peripheral storage means (64), user input means (63), display means (62), and processing means (58). The microprocessor can be programmed to process and interpret and display the ultrasound data in a variety of ways, including but not limited to A-mode imaging, B-mode imaging, M-mode imaging, Doppler audio with variable depth focus (gating), static colour Doppler, and Continuous wave Doppler. The preferred embodiment also provides a digital camera module (68), enabling users to record images of patients.

The wireless communications means can be used to save or download recorded patient data to an alternative system, such as but not limited to a medical records database operating on a personal computer, personal digital assistant (PDA), network server, or mainframe computer. The software on the system can include a client capable of connecting and synchronising to a medical records and practice management server, enabling a device registered to a physician to automatically download patient data from a practice management database to the device, removing the requirement for the physician to input patient data on the device. At the end of a patient session the device can upload data to a patient records database.

The handheld display and processing system provides an interface (56) with at least an always-on, single channel communications interface between the display and processing system and the ultrasonic probe. The interface is preferably a multi-master system, allowing either the display and processing system microprocessor or the ultrasound system microcontroller to wakeup the other system. The multi-master system allows either part of the system to initiate an ultrasound scan, providing maximum flexibility of operation.

The preferred embodiment's inclusion of a FPGA provides added flexibility in system expansion. The FPGA can be programmed to match the number of channels, communications speed, and even communications protocol of the probe. The FPGA can be programmed by the microprocessor (58) enabling future probes to provide updated FPGA firmware. Therefore, the system can be configured to match the operation of any probe design, even those invented in the future.

The handheld display and processing system provides non-volatile storage (64). An embodiment of the invention incorporates a secure data (SD) slot, enabling users to insert non-volatile flash memory cards. Another embodiment could incorporate a miniature hard disk. The user interface can be manipulated such that measurements taken by the device are recorded to non-volatile memory, along with a timestamp and other data identifying the patient.

It will be evident to the skilled hardware designer that the preferred embodiment can be implemented in many different forms depending on requirements. The forms can include standard microcontroller and DSP/FPGA components to a full custom ASIC design. Hence, the system could be constructed of numerous separate components (such as op-amps, A/D converters, D/A converters, digital signal processors, memory, displays, communications components etc), or could be comprised primarily of a mixed-mode application specific integrated circuit (ASIC) with a small number of support components.

The forgoing describes preferred forms of the present invention only. Modifications, obvious to those skilled in the art can be made thereto without departing from the scope of the invention.

Further, although the preferred embodiments are largely described in terms of medical/veterinary applications, the invention also finds use in other industrial applications, such as inspection of materials for internal damage/imperfections and such uses are encompassed within the scope of the present invention.

1-29. (canceled)

30. An ultrasound system including:
a. a handheld display and processing unit having:
(1) a display, and
(2) a first processor;
b. a probe unit having:
(1) an ultrasound transducer,
(2) transmit circuitry stimulating the ultrasound transducer to emit ultrasonic signals into a body to be imaged,
(3) receive circuitry receiving echo signals from the ultrasound transducer in response to echoes returned from a body to be imaged, and
(4) a position and/or orientation sensor:
(a) sensing relative or absolute position and/or orientation of the probe unit, and
(b) outputting the position and/or orientation of the probe unit as position data,
c. an interface:
(1) providing two way communication between the probe unit and the display and processing unit,
(2) including at least one communications channel transmitting the echo signals from the receive circuitry to the first processor;
wherein the first processor processes:
A. the echo signals from the receive circuitry, and
B. the position data,
for viewing on the display as an ultrasound image

31. The system of claim 30 wherein the probe unit includes a second processor combining the position data with the echo signals from the receive circuitry, the combined data being transmitted to the display and processing unit, wherein the first processor processes the combined data to display successively echo signals from the receive circuitry in correct spatial relation based on the received position data to form an ultrasound image.

32. The system of claim 30 wherein:
a. the probe unit further includes non-volatile storage media storing transducer calibration data characteristic of the ultrasound transducer,
b. the first or second processor reads the transducer calibration data and modifies the use of the echo signals from the receive circuitry based on the transducer calibration data so as to provide accurate image display for a variety of ultrasound transducers or probes.

33. The system of claim 32 wherein:
a. one or more of the processors is adapted to run a field calibration procedure for the ultrasound transducer,
b. the procedure includes the temporary attachment of a standard phantom to the probe unit, and
c. the results of the calibration procedure are stored in the non-volatile storage.

34. The system of claim 31 wherein:
a. the probe unit further includes non-volatile storage media storing sensor calibration data characteristic of the position and/or orientation sensor,
b. the first or second processor reads the sensor calibration data and modifies the use of the sensor data based on the sensor calibration data in order to provide accurate image display for a variety of ultrasound transducers or probes.

35. The system of claim 30 wherein the display and processing unit:
a. has at least substantially the same weight as the probe unit, and
b. a transmission cable connecting the display and processing unit to the probe unit, and carrying the communications channel, is of an appropriate length to provide a means to conveniently mechanically locate the system around the neck of a user.

36. The system of claim 30 wherein the display and processing unit includes user input apparatus comprising one or more of a scroll wheel, one or more push buttons, and a touchscreen.

37. The system of claim 30 wherein the probe unit includes secondary user input apparatus, comprising one or more of a scroll wheel and one or more push buttons.

38. The system of claim 37 wherein the secondary user input means allows for control of the depth of focus of the ultrasound signals.

39. The system of claim 30 wherein the display and processing unit includes:
a. a microphone,
b. a speaker,
c. software for recording and replaying user voice input, and
d. software adapted to associate the recorded user voice with an ultrasound image.

40. The system of claim 30 wherein the display and processing unit includes:
a. an integrated camera adapted to record photographic images, and
b. software adapted to associate a photographic image with an ultrasound image.

41. The system of claim 30 wherein the probe unit includes a coupling gel dispenser.

42. The system of claim 41 wherein the gel dispenser includes a replaceable gel cartridge.

43. The system of claim 30 wherein the probe unit includes:
a. the ultrasound transducer, which gathers ultrasound data, and
b. an acoustic transducer which gathers auscultation data,
wherein both auscultation data and ultrasound data are gathered without the need to change probes.

44. The system of claim 30 wherein the display and processing unit processes the outputs of the position and/or orientation sensor to allow movement of the probe unit to control a cursor on the display screen in a manner analogous to a computer mouse.

45. An ultrasound system including a handheld display and processing unit having:
a. a display;
b. an interface providing two way communication between an ultrasound probe unit and the display and processing unit;
c. a processor:
(1) processing digital image data and position and/or orientation data received from the ultrasound probe unit, and
(2) displaying successively received digital image data in correct spatial relation based on the position and/or orientation data to form an ultrasound image.

46. The handheld display and processing unit of claim 45 wherein the interface includes a plug and socket arrangement allowing the connection of alternative ultrasound probe units.

47. The handheld display and processing unit of claim 45 further including external data connectors for the connection of external devices.

48. A method for obtaining an ultrasound image comprising:
a. applying a probe unit to a body to be imaged, the probe unit including an ultrasound transducer and a position/orientation sensor;
b. moving the probe unit relative to the body;
c. receiving reflected ultrasound echoes as electrical signals from the ultrasound transducer,
d. translating the electrical signals into ultrasound scanline data,
e. receiving position and/or orientation data from the position/orientation sensor,
f. combining the position and/or orientation data with contemporaneously generated ultrasound scanline data,
g. transmitting the combined data to the display and processing unit,
h. displaying images generated from the ultrasound scanline data in correct spatial relation based on the received position and/or orientation data to form an ultrasound image.
